(19) United States
(12) Patent Application Publication
(10) Pub. No.: US 2010/0305011 A1
(43) Pub. Date: Dec. 2, 2010

(54) ANTI-OXIDATION AND/OR ANTI-CORROSION AGENT, LUBRICATING COMPOSITION CONTAINING SAID AGENT AND METHOD FOR PREPARING THE SAME

(75) Inventors: Pascal Frapin, Conflans Sainte Honorine (FR); Georges Issalatif, Montreuil (FR); Jean-Louis Mansoux, Parmain (FR)

Correspondence Address: YOUNG & THOMPSON, 209 Madison Street, Suite 500, Alexandria, VA, 22314, US

(73) Assignee: NYCO SA, Paris (FR)

(21) Appl. No.: 12/744,747

(22) PCT Filed: Nov. 28, 2008
(86) PCT No.: PCT/FR08/52156

§ 371 Date: May 26, 2010

(30) Foreign Application Priority Data
Nov. 28, 2007 (FR) 0759390

(51) Int. Cl. C10M 159/12; C07C 211/00

(52) U.S. Cl. 508/543; 564/305

(57) ABSTRACT

New anti-oxidation and/or anti-corrosion agents representing new products resulting from the reaction of a diphenyl amine compound (DPA) with a phenyl-α-naphthyl amine compound (PAN), possess improved properties, as compared to known additives. Lubricating compositions, in particular for turbines, containing the novel anti-oxidation and/or anti-corrosion agents, and a method for preparing the novel anti-oxidation and/or anti-corrosion agents, as well as the novel lubricating compositions are described.

### FIELD OF THE INVENTION

The present invention relates to the field of anti-oxidation and/or anti-corrosion agents, to be used mainly as additives to lubricating compositions.

### PRIOR ART

It is known for a long time that many organic or inorganic liquid, and solid materials used in industrial applications, as for example oils and greases, or liquids for use as a driving energy, may degrade and loss their qualities when exposed to oxidation.

Such an alteration of the initial properties is generally known as regards mineral oils and other lubricating compositions (or for transmitting energy), which during use are submitted to high temperatures, often in the presence of air.

This applies very especially to lubricating oils used in jet airplanes, which are employed under extreme conditions, with service temperatures that may reach over 200° C.

The lubricating oil oxidation stability is further reduced due to the dissolution of metals within these oils, under the extreme use conditions described hereabove. Indeed, dissolved metals may catalyze the oxidation-mediated degradation of the lubricants, thus resulting in a shortening of their lifetime.

That is why most of lubricants contain additives intended to prevent oxidation thereof.

Classically, antioxidant additives used in lubricating compositions belong to two major categories: (i) hindered phenols, relatively inefficient on esters, and (ii) aromatic organic amines, which have good performances on esters. This second type of antioxidant additives include for example additives marketed under the trade names Irganox® LO1, Irganox® LO6, and Naugalube® 348.

An antioxidant additive family which is sometimes used includes oligomers comprising combinations of units from diphenyl amine derivatives (DPA) and from phenyl-α-naphthyl amine derivatives (PAN).

A method for making antioxidant additives of the hereabove type has been described, for example in the U.S. Pat. No. 3,509,214 and U.S. Pat. No. 3,573,206. Both patents describe in particular how to prepare antioxidant additives by reacting equimolar amounts of N-phenyl-2-naphthyl amine and diphenyl amine in the presence of potassium permanganate. The ultimately resulting additive contained (a) 29.5% by weight of unreacted diphenyl amine, (b) 14.7% by weight of unreacted N-phenyl-2-naphthyl amine, (c) 35.3% by weight of N-phenyl-2-naphthyl amine dimers, and (d) 15.3% by weight of N-phenyl-2-naphthyl amine and diphenyl amine dimers, relative to the total weight of the additive composition.

Other methods for making antioxidant additives of a similar type, wherein an organic peroxide catalyst is used, have also been described. For example, the U.S. Pat. No. 3,492,233 describes additives prepared from diaryl amines in the presence of some organic peroxides.

The PCT application published under n^{o} WO 95/17488 describes lubricating compositions consisting in reaction products between N-aryl naphthyl amine compounds and diphenyl amine compounds in the presence of an organic peroxide, in an alkyl diphenyl amine to N-aryl naphthyl amine molar ratio of at least 1:1, and reaching up to 3:1 as experimentally illustrated. This document discloses the use, as an organic peroxide, of di-tert-butyl peroxide. The final resulting additive contained substantially a mixture of diphenyl amine homo-oligomers, and of oligomers of N-aryl naphthyl amine and diphenyl amine. Moreover, the final resulting additive is free of any potassium permanganate catalyst or of any reduction product thereof.

The PCT application published under n^{o} WO 95/16765 also describes antioxidant compositions consisting in reaction products resulting from the reaction between N-aryl naphthyl amine compounds and diaryl amine compounds in the presence of an organic peroxide, in a diaryl amine to N-aryl naphthyl amine molar ratio of at least 1:1 and reaching up to 3:1 as experimentally illustrated. This document discloses the use, as an organic peroxide, of di-tert-butyl peroxide. The reaction is carried out in the absence of potassium permanganate. The final resulting additive contained preferably at least 30 mol % of diaryl amines having reacted with the solvent as dehydrocondensation products, at least 10 mol % of diaryl amines as homo-oligomers and at least 35 mol % of diaryl amines as diphenyl amine and N-aryl naphthyl amine cross-oligomers.

According to this document, any use of potassium permanganate should be avoided, due to the fact that the permanganate catalysts result in the formation of oligomer products derived from diamines and possessing reduced antioxidant effects.

In the French patent application published under n^{o} FR 2 832 417, lubricating compositions have also been described, comprising an antioxidant additive having three components, respectively (1) a substituted diphenyl amine (DPA), (2) a phenyl-α-naphthyl amine (PANA) and (3) an oligomeric antioxidant obtained by reacting a DPA with a PANA. Conditions for preparing the antioxidant additive are similar to, if not the same as those described for preparing the additives disclosed in the PCT applications n^{o} WO 95/17488 and n^{o} WO 95/16765 as discussed hereabove. In particular, the additive described in the application FR 2 832 417 was obtained in the presence of one or more organic peroxide(s), di-tert-butyl peroxide being the only one organic peroxide given as an example.

As a whole, antioxidant additives that are known for lubricating compositions have good properties improving the stability of these compositions and extending their lifetime.

However, there is still a need in the state of the art for antioxidant additives that would be alternatives or improved developments, as compared to known additives. There is also a need for novel methods for preparing these antioxidant additives.

### SUMMARY OF THE INVENTION

The present invention provides a method for preparing new anti-oxidation and/or anti-corrosion agents representing new products resulting from the reaction of a diphenyl amine compound (DPA) with a phenyl-α-naphthyl amine compound (PAN).

The novel anti-oxidation and/or anti-corrosion agents prepared according to the method of the invention do possess improved properties, as compared to known additives.

The present invention also provides lubricating compositions, in particular for turbines, comprising the novel anti-oxidation and/or anti-corrosion agents hereabove, said lubricating compositions possessing improved stability properties, as compared to known lubricating compositions.

### DESCRIPTION OF THE FIGURES

FIG. 1 illustrates a chromatogram of the anti-oxidation agent of the invention in the supercritical phase.

### DETAILED DESCRIPTION OF THE INVENTION

The applicant strove to develop new anti-oxidation and/or anti-corrosion agent-containing compositions, based on a product resulting from the reaction of at least one diphenyl amine (DPA) and at least one phenyl-α-naphthyl amine (PAN), which would possess an outstanding ability to stabilize the characteristics of oil compositions as to the transmission of energy and of lubricating oil compositions, especially lubricating oil compositions that are intended to be used at high temperature, such as oils for aircraft engine turbines.

After a long-lasting research process, an anti-oxidation and/or anti-corrosion agent has been developed according to the present invention, the specific formulation of which imparts to the same outstanding reduction properties of unwanted oxidation and deposit formation phenomena which do occur upon using industrial oils, especially under high temperature conditions and in the presence of oxygen.

It is therefore an object of the present invention to provide a method for preparing an anti-oxidation agent for use in lubricating compositions, and more particularly in lubricating compositions for aircraft engine turbines.

Surprisingly, the applicant has shown that an anti-oxidation agent with a good quantitative and qualitative formulation as regards monomers and oligomers imparting to the same outstanding industrial oil stabilizing properties could be obtained from a diphenyl amine compound (DPA) of formula (I) and a phenyl-α-naphthyl amine compound (PAN) of formula (II), according to a specific DPA to PAN molar ratio and by using potassium permanganate as the reactant for the condensation reaction.

It is an object of the present invention to provide a method for preparing an anti-oxidation and/or anti-corrosion agent for a lubricating oil, consisting in the following steps of:
a) reacting
   (i) a diphenyl amine compound (DPA) of following formula (I):

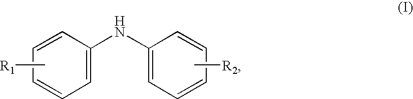

wherein R₁ and R₂ groups, independently from each other, represent a linear or a branched alkyl group comprising from 4 to 12 carbon atoms,
with
(ii) a phenyl-α-naphthyl amine compound (PAN) of following formula (II):

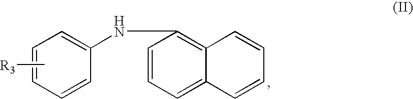

wherein R₃ group represents a linear or a branched alkyl group comprising from 4 to 12 carbon atoms,
in a compound (I) to compound (II) molar ratio ranging from 2:1 to 5:1, preferably from 2.5:1 to 4:1, in the presence of potassium permanganate, in a solvent and at a temperature ranging from 85° C. to 150° C.;
b) cooling the reaction mixture to a temperature of at most 80° C.;
c) filtering the reaction mixture cooled in step b).

As used herein, the term “alkyl” is intended to mean a linear or branched chain of a monovalent, saturated hydrocarbon radical, with the specified number of carbon atoms. In a branched alkyl group, the linear hydrocarbon chain is substituted by one or more alkyl groups. Alkyl groups include in particular butyl, pentyl, hexyl and octyl groups.

In some embodiments of the anti-oxidation agent, R₁ and R₂ groups of the diphenyl amine compound (DPA) are the same.

Diphenyl amine compounds (DPA) include dioctylphenyl amine, didecylphenyl amine, didodecylphenyl amine or dihexylphenyl amine.

In some embodiments of the anti-oxidation agent, R₁ and R₂ groups of the diphenyl amine compound (DPA) each represent an octyl group and DPA is di-octyl diphenyl amine or DODPA, well known from the one skilled in the art.

Phenyl-α-naphthyl amine compounds encompass in particular para-tert-octylphenyl-α-naphthyl amine, para-tert-dodecylphenyl-α-naphthyl amine and para-tert-butyl-α-naphthyl amine.

In some embodiments of the anti-oxidation agent, R₃ group of the phenyl-α-naphthyl amine compound (PAN) consists of an octyl group and PAN is octyl-phenyl-α-naphthyl amine or N-(para-tert-octylphenyl)-α-naphthyl amine, also referred to as OPAN, well known from the one skilled in the art.

Generally speaking, DPA compounds of formula (I) and PAN compounds of formula (II) are products that are widely commercially available.

Preferably, a DPA compound of formula (I) is used wherein R₁ and R₂ groups each consists of an octyl group and DPA is di-octyl phenyl amine or DODPA, well known from the one skilled in the art.

Preferably, a PAN compound of formula (II) is preferably used, wherein R₃ group consists of an octyl group and PAN is octyl-phenyl-α-naphthyl amine or N-(para-tert-octylphenyl)-α-naphthyl amine, also referred to as OPAN, well known from the one skilled in the art.

Preferably, in step a), the reaction mixture comprises the DPA compound, the PAN compound and potassium permanganate in dissolved form in an organic solvent adapted to the reaction temperature. Solvents to be suitably used encompass solvents based on aliphatic hydrocarbon compounds, including alkanes comprising from 6 to 16 carbon atoms in a cyclic, branched or linear structure. A petroleum type solvent may be used for example like the Exxsol DSP 10:140 solvent marketed by the ExxonMobil Chemical company.

The weight ratio [PAN+DPA+potassium permanganate reactants]/[solvent] is of about 1.

Preferably, at least step a) of the method is carried out under an inert gas atmosphere strongly depleted of oxygen so as to avoid unsuitable oxidation reactions. Classically, step a) is carried out within a reactor under nitrogen or argon atmosphere.

In step a), the DPA to PAN molar ratio is preferably ranging from 3:1 to 3.5:1, which is the optimal DPA to PAN molar ratio range for obtaining an anti-oxidation agent having a quantitatively and qualitatively also optimal composition as regards monomer(s) and oligomers imparting industrial oil stabilizing high properties.

In step a), the reaction temperature is advantageously of at most 100° C. More preferably, the reaction temperature is advantageously of at least 30° C.

Advantageously, step a) is a step carried out under reflux conditions.

Advantageously, in step a), compounds of formula (I) and compounds of formula (II) in the solvent are first added, then after a pre-heating period, the potassium permanganate catalyst is added thereto so as to initiate the condensation reaction strictly speaking. Said pre-heating period is carried out for the time required to bring the initial reaction mixture with no permanganate reactant up to the suitable reaction temperature. The pre-heating period time may range from 1 minute to 1 hour, depending on the selected reaction conditions, very especially depending on the system used and on the initial reaction mixture volume.

For ensuring optimal reaction conditions, step a) may comprise the following sub-steps of:
a1) providing a reactor containing the selected solvent in a suitable amount;
a2) adding the suitable amounts of each of DPA compound of formula (I) and PAN compound of formula (II);
a3) optionally, arranging the reactor under an oxygen-depleted atmosphere, for example by injecting nitrogen or argon;
a4) effecting a pre-heating of the reaction medium obtained at the end of step a2) or step a3) at a temperature ranging from 55° C. to 85° C., preferably from 60° C. to 80° C.;
a5) adding to the reaction mixture obtained at the end of step a4) the right amount of potassium permanganate;
a6) increasing the reaction mixture temperature up to the chosen reaction temperature;
a7) maintaining the reaction medium at the selected reaction temperature, for the time required to obtain the final desired content of DPA of formula (I).

The sequence of operations of steps a3) to a5) hereabove, or as an alternative of steps a4) and a5) hereabove, has no importance, knowing that optimal reaction conditions are obtained when following initial order of from a1) to a7) is considered.

Most preferably, the reaction medium is maintained in step a), and more particularly in step a7), at a temperature ranging from 110° C. to 125° C.

In step a7) the oxidation-mediated condensation reaction is initiated and it is important to closely regulate the temperature value in the reaction medium, because it is an exothermic reaction.

Step a) duration is advantageously of at least 5 hours and is typically of at least 10 hours. Step a) duration is typically of at most 30 hours, and is typically of at most 20 hours, depending on the chosen reaction conditions, in particular on the chosen temperature conditions. Generally speaking, step a) duration is determined by step a7) duration, which is a step during which the condensation reaction strictly speaking is effected.

In step b) of the method, the reaction mixture is cooled to a temperature of at most 80° C., for example by simply turning the heating means off.

In step c), the reaction mixture is filtered so as to remove the possible deposits that have been produced during the previous steps. Advantageously, a fine filtration is carried out so as to reduce the deposit content to a maximum value of 1 mg per liter of reaction mixture, in accordance with the Standard FTM-S-791-3010—Federal Test

Method for example, as defined by the American government.

In some embodiments of step c), filtration strictly speaking may be followed with a washing operation of the reaction medium with an aqueous solution, typically demineralized water, so as to remove from the solvent the possible residual impurities. Thereafter the aqueous solution is removed, for example just by drawing it off, prior to carrying out the desolvation step d).

As already previously described, the anti-oxidation agent of the invention may present as a powder.

For preparing the anti-oxidation agent of the invention as a powder, the hereabove mentioned method comprises the following additional step consisting in:
d) removing any residual solvent, so as to obtain the anti-oxidation and/or anti-corrosion agent as a powder.

In step d), the residual solvent may be removed by means of any known desolvation method, including through desolvation by vacuum heating, the operating conditions being adapted to the type of solvent used. For example, desolvation under vacuum may be carried out at a temperature ranging from 140° C. to 170° C., advantageously from 150° C. to 160° C. If needed, the solvent removal may be completed by flushing the reaction product with a neutral gas, for example nitrogen or argon.

Preferably, the solvent content of the reaction product obtained at the end of step d) is adjusted to a value that is lower than 50 mg of solvent per kg of the final reaction product.

In the method of the invention, using a compound (I) to compound (II) molar ratio of at least 2:1 and at most 5:1 enables in particular to adjust the unreacted monomeric DPA amount that is retrieved in the final product of the method, i.e. the anti-oxidation agent of the invention. Thus, with a DPA to PAN molar ratio of less than 2:1, a final product is obtained, which unreacted DPA end content is lower than 20% by weight, relative to the desolvated final product total weight. In addition, with a DPA to PAN molar ratio higher than 5:1, a final product is obtained, which unreacted DPA end content is higher than 30% by weight, relative to the desolvated final product total weight.

Depending on the method, a molar ratio of at least 2.5:1 and at most 4:1 is preferably used.

Thanks to an advantageous characteristic, a potassium permanganate to [compound (I)+compound (II)] molar ratio is used in step a), that is of at least 0.25:1 and at most 0.35:1. The potassium permanganate to [compound (I)+compound (II)] molar ratio is preferably of at least 0.28:1. The potassium permanganate to [compound (I)+compound (II)] molar ratio is preferably of at most 0.33:1.

Selecting an optimal permanganate to DPA and PAN initial products molar ratio is important for obtaining a final product possessing the expected qualitative oligomeric composition.

With a potassium permanganate to [compound (I)+compound (II)] molar ratio of less than 0.25, a final reaction product is obtained, which is characterized in particular by an unreacted DPA content higher than 30% by weight, relative to the desolvated final product total weight.

With a potassium permanganate to [compound (I)+compound (II)] molar ratio higher than 0.35, a final reaction product is obtained, which is particularly characterized by an unreacted DPA content of less than 20% by weight, relative to the desolvated final product total weight.

To obtain an anti-oxidation agent final product possessing industrial oil stabilizing optimal properties, a potassium permanganate to [compound (I)+compound (II)] molar ratio ranging from 0.30:1 to 0.33:1 is advantageously used in step a) of the method.

At the end of step d) of the method, an anti-oxidation agent of the invention is obtained, which presents as a powder.

The anti-oxidation agent of the invention may be used for preparing an antioxidant composition in a liquid form.

In a first embodiment of the method for making a liquid antioxidant composition from an anti-oxidation agent of the invention, the powdered anti-oxidation agent obtained at the end of step d) of the method hereabove is added in a suitable amount to a suitable volume of oil, preferably a synthetic ester type oil.

In a second embodiment of the method for making a liquid antioxidant composition from an anti-oxidation agent of the invention, said antioxidant composition is prepared according to a method comprising the steps a) to c) of the hereabove described method, said method further comprising the additional following steps consisting in:
d) preparing a mixture with (i) the filtered reaction medium obtained at the end of step c) and (ii) a suitable amount of an oil, preferably a synthetic ester type oil;
e) removing the solvent, so as to obtain an antioxidant composition in a liquid form.

As previously mentioned, in an antioxidant composition in a liquid form of the invention, whatever its preparation mode, the anti-oxidation agent is present in the oily liquid in an amount ranging from 10% to 60% by weight of the powdered anti-oxidation agent, and preferably in an amount ranging from 20% to 50% by weight, relative to the liquid composition total weight. Most preferably, the anti-oxidation agent content does range from 25% to 35% by weight, relative to the liquid antioxidant composition total weight.

As already generally explained, the anti-oxidation agent of the invention, or as an alternative an antioxidant composition such as defined hereabove, is intended to be used as an additive to industrial oils, in particular to energy transmitting oils for hydraulic systems and to lubricating oils, typically to oils for aircraft engine turbines.

In particular, the anti-oxidation agent of the invention and the antioxidant compositions hereabove are intended to be used as synthetic ester-based additives to lubricants for gas turbines.

It is also an object of the present invention to provide an anti-oxidation and/or anti-corrosion agent obtained by oligomerizing:
(i) a diphenyl amine compound (DPA) of following formula (I):

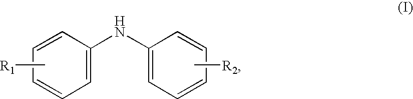

wherein R₁ and R₂ groups, independently from each other, represent a linear or a branched alkyl group comprising from 4 to 12 carbon atoms, and
(ii) a phenyl-α-naphthyl amine compound (PAN) of following formula (II):

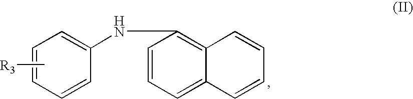

wherein R₃ group represents a linear or a branched alkyl group comprising from 4 to 12 carbon atoms,
said anti-oxidation and/or anti-corrosion agent comprising:
   (a) at least 20% by weight of diphenyl amine of formula (I),
   (b) from 25% to 35% by weight of oligomers as dimers,
   (c) from 25% to 35% by weight of oligomers as trimers,
   (d) from 10% to 15% by weight of oligomers as tetramers,
   (e) from 3% to 8% by weight of oligomers as pentamers, and
   (f) less than 1% by weight of phenyl-α-naphthyl amine of formula (II),
   the weight percentages being expressed relative to the component (a) to (f) total weight, and
   said anti-oxidation agent being substantially free of organic peroxide or free of any organic peroxide decomposition product.

It has been shown according to the present invention that the (a) to (f) component quantitative and qualitative composition of the anti-oxidation agent defined hereabove enables to impart to said agent properties for highly stabilizing industrial oils, and especially oils for aircraft engine turbines. It has been in particular demonstrated that the anti-oxidation agent of the invention enables to significantly reduce the acid number variation of oils to which such agent is added, which proves the stabilizing effect on these oils submitted to extreme use conditions. It has also been shown that said anti-oxidation agent enables to significantly reduce the viscosity variation of oils to which it is added, which proves that the anti-oxidation agent of the invention enables to maintain the energy-transfer and lubricating properties of these oils.

Thus, by using the anti-oxidation agent of the invention, or a liquid antioxidant composition of the invention, it becomes possible to extend the lifetime of industrial oils used under temperature and pressure extreme conditions, which is very profitable both as regards the use security and the economical approach.

DPA and PAN compounds used in the method are selected from DPA and PAN compounds as previously defined in the present specification.

An amount of at least 20% by weight of diphenyl amine of formula (I) in the anti-oxidation agent is favorable to the global effect of said agent on the maintenance, together with the lifetime, of the industrial oil properties to which such agent was added. Another advantage of such a DPA amount lies in the optimal molecular distribution of said agent, with in particular a limited higher molecular weight oligomer content. Indeed, high molecular weight oligomers have a thickening effect on oil, which necessarily requires the use of a fluid ester base, with as a consequence a high evaporation and a swelling of the elastomers.

Advantageously, the content of diphenyl amine of formula (I) is at most of 30% by weight, relative to the component (a) to (f) total weight of said agent.

Advantageously, the dimer content dos range from 28% to 33% by weight, relative to the component (a) to (f) total weight of said agent.

Advantageously, the dimers comprise predominantly DPA of formula (I) and PAN of formula (II)-crossed dimers. It is necessary to start from both components so as to obtain mono-, di-, tri-, and tetramers, distributed into homo- and hetero-oligomers.

Advantageously, the trimer content does range from 28% to 33% by weight, relative to the component (a) to (f) total weight of the anti-oxidation agent.

Advantageously, the tetramer content does range from 11% to 14% by weight, relative to the component (a) to (f) total weight of the anti-oxidation agent.

Advantageously, the pentamer content does range from 3.5% to 5.5% by weight, relative to the component (a) to (f) total weight of the anti-oxidation agent.

Generally speaking, the anti-oxidation agent qualitative and quantitative composition of the invention may be easily determined by the person skilled in the art, by any known technique. For example, the person skilled in the art may use a high performance liquid chromatography (HPLC) or a vapor-phase chromatography technique. The person skilled in the art may also use a supercritical fluid chromatography (SFC).

According to another characteristic of the anti-oxidation agent of the invention, said agent is substantially free of, or, as an alternative, totally free of any detectable organic peroxide content, including alkyl peroxide, and specifically, di-tert-butyl peroxide, as well as of any decomposition product thereof, as for example ter-butanol.

According to a further characteristic of the anti-oxidation agent of the invention, said agent may contain detectable traces of potassium permanganate or of reduction products thereof. Their content may be measured by flame ionization spectrometry (ICP).

It is also a further object of the present invention to provide antioxidant compositions in a liquid form containing the anti-oxidation agent such as defined in the present specification, or obtained according to the previously detailed method. Preferably, the antioxidant compositions of the invention do present in the form of an oily liquid, wherein components (a) to (f) are dissolved into an oil.

Advantageously, according to this embodiment, the powdered anti-oxidation agent is dissolved into a synthetic ester-based oil selected from oils well known from the person skilled in the art in the field of lubricants for turbines. It may be in particular a polyol diester- and ester-based oil of a known type.

Advantageously, according to this embodiment, the anti-oxidation agent is present in the oily liquid in an amount ranging from 10% to 60% by weight of the powdered anti-oxidation agent, and preferably in an amount of 30% by weight of the anti-oxidation agent, relative to the antioxidant composition total weight.

Characteristics of the Liquid Antioxidant Compositions:

a) According to an additional characteristic, the antioxidant composition in a liquid form, in one embodiment wherein the antioxidant agent content is of about 30% by weight, ranging from 20% to 50% by weight, relative to the liquid antioxidant composition total weight, presents as a clear liquid with a density of about 0.989 kg/dm³ at 20° C. according to standard ISO 12185 (Crude Petroleum and Petroleum products—Determination of density—Oscillating U-tube method). Most preferably, the anti-oxidation agent content does range from 25% to 35% by weight, relative to the liquid antioxidant composition total weight.

b) According to other characteristics, said antioxidant composition in a liquid form does possess kinematic viscosity values of about 14.8 mm²/s at 100° C. and of about 160 mm²/s at 40° C., such as measured with the method according to standard ISO 3104 (Petroleum products. Transparent and opaque liquids.—Determination of kinematic viscosity and calculation of dynamic viscosity). According to still further characteristics, said antioxidant composition in a liquid form does possess a Cleveland Open Cup (COC) flash point value of about 264° C., such as measured with the method according to standard ISO 2592 (Determination of flash and fire points—Cleveland open cup).

c) According to a further characteristic, said antioxidant composition in a liquid form does possess an acid number of about 0.03 mg KOH/g, such as measured with the method according to standard ARP 5088 (Total Acid Number (TAN) Measurement for Oil Samples).

It is also an object of the present invention to provide lubricating compositions having an improved deposition resistance as well as an improved oxidation stability, comprising a suitable amount of an anti-oxidation agent such as defined hereabove.

In some embodiments of the lubricating compositions of the invention, said anti-oxidation agent presents in the form of a liquid antioxidant composition such as described hereabove, which composition may comprise, as already stated, an anti-oxidation agent content ranging from 10% to 60% by weight of said anti-oxidation agent, relative to said antioxidant composition total weight.

In a lubricating composition according to the invention, the anti-oxidation agent content is always expressed as the anti-oxidation agent end content per se, and not as the antioxidant content of a composition itself comprising said anti-oxidation agent. As an example, a lubricating composition comprising 2.5% by weight of an anti-oxidation agent may be obtained (i) by directly adding to the lubricating composition the suitable amount of anti-oxidation agent (for example 2.5 g of anti-oxidation agent added to 97.5 g of lubricating composition), or (ii) by adding to the lubricating composition the right amount of an antioxidant composition comprising said anti-oxidation agent (for example 25 g of an antioxidant composition containing 10% by weight of an anti-oxidation agent added to 75 g of a lubricating composition).

As illustrated in the examples, a lubricating composition comprising an antioxidant additive such as defined in the present specification, or that may be obtained through the method of the invention, has an acid number variation after an accelerated ageing process according to standard FTM-S-791-5308 (test conditions: 72 hours at 204° C.), such as according to standard ARP 5088, that is lower than 1.0 mg of KOH/g, preferably lower than 0.9 mg of KOH/g. In some embodiments, said lubricating composition has an acid number variation that is lower than 0.9 mg of KOH/g, 0.8 mg of KOH/g, 0.7 mg of KOH/g, or even lower than 0.65 mg of KOH/g. These characteristics are confirmed in particular for lubricating compositions comprising 2.5% by weight of an antioxidant additive of the invention, relative to said lubricating composition total weight.

As illustrated in the examples, a lubricating composition comprising an antioxidant additive such as defined in the present specification, or that may be obtained through the method of the invention, has a kinematic viscosity variation after an accelerated ageing process according to standard FTM-S-791-5308 (72 h at 204° C.), such as according to standard ISO 3104, that is lower than 14%, preferably lower than 10%. In some embodiments, said lubricating composition has a kinematic viscosity variation that is lower than 13%, 12%, 11%, 10%, 9% or even lower than 8.5%. These characteristics are confirmed in particular for lubricating compositions comprising 2.5% by weight of an antioxidant additive of the invention, relative to said lubricating composition total weight.

Advantageously, a lubricating composition of the invention comprises an anti-oxidation agent in an amount such as defined hereabove ranging from 0.1% to 10% by weight of the powdered agent, relative to said lubricating composition total weight. In embodiments wherein the anti-oxidation agent which is added to the lubricating composition presents in the form of a liquid as previously described, the anti-oxidation agent content in the lubricating composition is calculated based upon the initial powder amount of said anti-oxidation agent.

Preferably, a lubricating composition of the invention comprises an antioxidant agent in an amount ranging from 0.5% to 5% by weight, and most preferably in an amount ranging from 1.5% to 3% by weight, relative to said lubricating composition total weight.

Generally speaking, the anti-oxidation agent may be added to various types of industrial oils, including oils suitable for transferring energy commonly used in transmission devices, and oils for lubrication.

In some embodiments of lubricating compositions of the invention, the anti-oxidation agent is added to lubricating oils based upon synthetic esters well known from the person skilled in the art, in particular in the field of lubricants for aircraft engine turbines.

Lubricating oils may be used for example based upon esters derived from mono-hydroxylated alcohols and mono-carboxylic acids, or from mono-hydroxylated alcohols and dicarboxylic acids. Such esters are well known from the person skilled in the art. They are described for example in the U.S. Pat. No. 3,432,433. Alcohols and acids used for preparing the esters may contain from one to six functional groups, which enables to produce mono-, di-, tri- and tetra-, penta- and hexa-esters. Included are alcohol esters, diols, triols and pentaerythritols, said alcohols or polyols having from 2 to 20 carbon atoms, and mono- and di-carboxylic acids having from 2 to 20 carbon atoms, preferably from 4 to 12 carbon atoms. Polyols include trimethylolpropane, pentaerythritol, dipentaerythritol, neopentylglycol, tripentaerythritol, di-TMP and their mixtures.

Esters that may be contained in a lubricating composition of the invention include octyl acetate, decyl acetate, octadecyl acetate, methyl myristate, butyl stearate, methyl oleate monoesters, as well as dibutyl phthalate, di-octyl adipate, di-2-ethylhexyl azelate and ethylhexyl sebacate polyesters. The basic oil of the polyol ester type may be an oil prepared from technical pentaerythritol or from trimethylol propane with a mixture of carboxylic acids having from 4 to 12 carbon atoms. Technical pentaerythritol is a mixture which comprises approximately from 85% to 92% by weight of monopentaerythritol and from 8% to 15% by weight of dipentaerythritol.

A classically marketed technical pentaerythritol comprises approximately 88% by weight of monopentaerythritol and approximately 12% by weight of dipentaerythritol, relative to said ester-type basic oil total weight. Technical pentaerythritol may further contain some amount of tri- and tetra-pentaerythritol which are usually generated as byproducts during the production process of technical pentaerythritol.

In a lubricating composition of the invention, the anti-oxidation agent may be used in combination with other additives, such as detergents, antifoaming agents, anti-wear agents, extreme pressure additives, hydrolysis stabilizing agents, fillers or viscosity modifying agents, such additives being well known from the person skilled in the art and commonly available on the market.

Generally speaking, a lubricating composition of the invention, due to the presence of the anti-oxidation agent defined in the present specification, consists in a composition intended to be used under extreme use conditions, in particular under extreme temperatures, for example within the −50° C. to +250° C. range, and which would possess outstanding stability properties, including oxidation stability properties, viscosity stability characteristics, chemical degradation stability and deposit formation reduction properties. Chemical degradation stability is in particular characterized by an acid number low variation, even after an extended time of use of the lubricating composition under high pressure and high temperature use conditions.

As an illustration of the stabilizing properties of a lubricating composition of the invention, a lubricating composition may be mentioned, based on technical pentaerythritol ester and comprising from 1.5 to 3% by weight of an anti-oxidation agent of the invention, for which an acid number variation of less than 1 mg KOH/g and a kinematic viscosity variation of less than 8% have been determined, according to the oxidation corrosion test as defined by FTM-S-791-5308.

The present invention will be now illustrated by means of the following examples.

### EXAMPLES

### Example 1

### Preparation of an Anti-Oxidation Agent in a Liquid Form

In a Pyrex flask fitted with a stirring rod in stainless steel, with a thermometric jacket, a nitrogen bubbler, a Dean stark apparatus and a cooler:

Fill
20 g of OPAN
80 g of DODPA
117 g of solvent (Exxsol DSP 100:140)

The reaction proceeds under inert atmosphere (nitrogen).

Heat and stir (control the temperature increase)

At a temperature of about 70° C., add 14.6 g of KMnO4 at 1 time.

Gradually increase the temperature until the reaction begins (125° C.) and reflux is obtained in the dean stark apparatus (warning: exothermic reaction).

Maintain these conditions until a residual DODPA content of about 20% is obtained.

At the end of the reaction, filter the reaction mixture on a folded filter, and then on a 1.2 μm-filter membrane, until a deposit content of less than 2 mg/l is obtained. The K and Mn content is then null.

Solvent is removed under vacuum and using a heat source. Depending on the chosen solid or liquid (by dilution) end form and on the material used, the desolvation end temperature may vary (for example, by dilution, final temperature range=150-160° C. under 2 to 3 mm/Hg).

Measure the solvent residual content: should be null.

### Example 2

### Composition Analysis of the Anti-Oxidation Agent of the Invention

An analysis was performed through a supercritical chromatography method, by carrying out the following protocol:

Preparation of a 20% DODPA Standard Solution:

80% of copolymer with no DODPA monomer and 20% of DODPA diluted 100 times with heptane are prepared, thereafter 5 μl are injected in a supercritical fluid chromatography (SFC).

Preparation of the Reaction Mixture to Test as a Solution:

A sample of the oxidizing reaction medium (−50% in DSP 100:140) is collected, which is diluted 50 times with ethyl acetate, thereafter 5 μl are injected in a SFC.

Free DODPA UV-Assay at 268 nm:

DODPA peak areas in both cases are compared to each other:

% DODPA=peak area of the DODPA to test*20/peak area of the standard DODPA

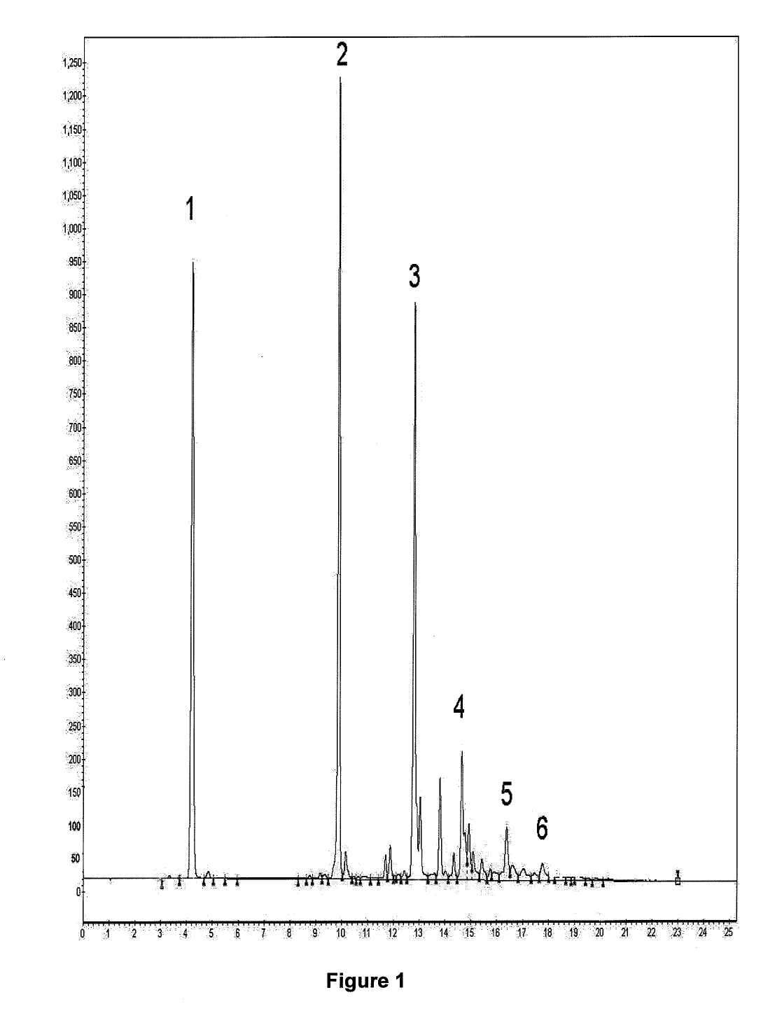

The results are given on the chromatogram on FIG. 1, as well as in Table 1 hereunder.

TABLE 1
qualitative and quantitative composition of the
anti-oxidation agent according to the invention

| | Anti-oxidant of the |
|---|---|
| Component type | invention* |
| DODPA monomer | 23.1 |
| OPAN monomer | ND** |
| Dimers | 30.6 |
| Trimers | 29.2 |
| Tetramers | 12.4 |
| Pentamers | 4.7 |

*weight percentage relative to the anti-oxidation agent total weight
**Non Detectable

### Example 3

### Preparation of a Lubricating Composition Comprising the Anti-Oxidation Agent of the Invention

The required amounts of ester and additives are weighted in a beaker, amongst which the additive of the invention.
Example of a composition (weight %)

| | |
|---|---|
| Ester | 94.61 |
| Additive of the invention | 2.5 |
| Other additives | 2.89 |

Heat under stirring up to a temperature of 110° C. and until complete dissolution of the additives.

Filter.

### Example 4

### Comparative Results of the Properties of a Lubricating Composition of the Invention with a Commercial Oil Containing a Different Anti-Oxidation Agent

4.1. Comparison Between the Anti-Oxidant Additive-Containing Oil Compositions

A special turbine oil is formulated, having following composition:

94.45% of a polyol ester (C5, C7, C8-C10+pentaerythritol and dipentaerythritol acid ester)

3% of a triaryl phosphate antiwear additive

2.5% of an anti-oxidant

0.05% of a corrosion inhibitor.

The comparative commercial oil is BP Turbo Oil 2197 marketed by Air BP, the test results being those mentioned in the sales literature.

4.2. Test Protocol

Accelerated ageing was performed via the oxidation corrosion test according to standard FTM-S-791-5308, under the standard test conditions (72 hours at 204° C.).

4.3. Results

Acid Number Variation

Acid number values were measured for each of both compared lubricating compositions, respectively prior to and after having simulated the ageing process of these compositions. Acid number values were measured according to standard ARP 5088.

Results show that the acid number variation value is of about 1.0 mg of KOH/g for the comparative lubricating composition, while the acid number variation value is of about 0.6 mg of KOH/g for the lubricating composition containing the anti-oxidation agent according to the invention.

These results show that the lubricating composition containing an anti-oxidation agent according to the invention does possess a better stability and less degradation, as compared to the comparative lubricating composition, which comprises a different anti-oxidation agent also derived from DPA and PAN.

Kinematic Viscosity Variation

Kinematic viscosity values were measured for each of both compared lubricating compositions, respectively prior to and after having simulated the ageing process of these compositions. Kinematic viscosity values were measured according to standard ISO 3104.

Results show that the kinematic viscosity value variation is of about 14.8% for the comparative lubricating composition, while the kinematic viscosity value variation is of about 8.3% for the lubricating composition containing the anti-oxidation agent according to the invention.

These results show that the lubricating composition containing an anti-oxidation agent according to the invention do possess a better stability and less degradation, as compared to the comparative lubricating composition, which comprises a different anti-oxidation agent also derived from DPA and PAN.

1. A method for preparing an anti-oxidation and/or anti-corrosion agent for a lubricating oil, consisting in the following steps of:
a) reacting
(i) a diphenyl amine compound (DPA) of following formula (I): 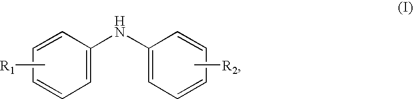
wherein R₁ and R₂ groups, independently from each other, represent a linear or a branched alkyl group comprising from 4 to 12 carbon atoms,
with
(ii) a phenyl-α-naphthyl amine compound (PAN) of following formula (II): 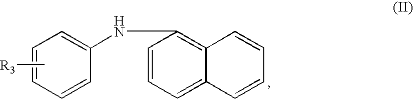
wherein R₃ group represents a linear or a branched alkyl group comprising from 4 to 12 carbon atoms,
in a compound (I) to compound (II) molar ratio ranging from 2:1 to 5:1 in the presence of potassium permanganate, in a solvent and at a temperature ranging from 85° C. to 150° C.;
b) cooling the reaction mixture to a temperature of at most 80° C.; and
c) filtering the reaction mixture cooled in step b).

2. A method according to claim 1, wherein for the diphenyl amine compound (DPA) of formula (I), R₁ and R₂ groups each represent an octyl group.

3. A method according to claim 1, wherein for the phenyl-α-naphthyl amine compound (PAN) of formula (II), R₃ group represents an octyl group.

4. A method according to claim 1, wherein in step a), a potassium permanganate to [compound (I)+compound (II)] molar ratio is used, ranging from 0.25:1 to 0.35:1, preferably from 0.28:1 to 0.33:1.

5. A method according to claim 1, which comprises the following additional step consisting in:
d) removing any residual solvent, so as to obtain the anti-oxidation and/or anti-corrosion agent, as a powder.

6. A method according to claim 5, which comprises an additional step e) of adding an oil to the anti-oxidation and/or anti-corrosion agent obtained in step d).

7. A method according to claim 6, wherein said oil consists in a synthetic ester type oil.

8. A method according to claim 1, which comprises the following additional steps of:
d) preparing a mixture with (i) the filtered reaction medium obtained at the end of step c) and (ii) a suitable amount of an oil, preferably a synthetic ester type oil;
e) removing the solvent, so as to obtain an anti-oxidation agent in a liquid form.

9. An anti-oxidation and/or anti-corrosion agent that may be obtained through the method according to claim 1, said anti-oxidation agent and/or said agent anti-corrosion comprising:
(a) at least 20% by weight of diphenyl amine of formula (I),
(b) from 25% to 35% by weight of oligomers as dimers,
(c) from 25% to 35% by weight of oligomers as trimers,
(d) from 10% to 15% by weight of oligomers as tetramers,
(e) from 3% to 8% by weight of oligomers as pentamers, and
(f) less than 1% by weight of phenyl-α-naphthyl amine of formula (II),
the weight percentages being expressed relative to the component (a) to (f) total weight, and
said anti-oxidation agent being substantially free of organic peroxide or free of any organic peroxide decomposition product.

10. An antioxidant composition having an anti-oxidation agent content according to claim 9 ranging from 10% to 60% by weight of said anti-oxidation agent, relative to said composition total weight.

11. A lubricating composition comprising
an anti-oxidation agent that may be obtained through the method according to claim 1, having an acid number variation after an accelerated ageing process according to standard FTM-S-791-5308, such as according to standard ARP 5088, of less than 1.0 mg of KOH/g, preferably of less than 0.9 mg of KOH/g.

12. A lubricating composition comprising an anti-oxidation agent that may be obtained through the method according to claim 1, having a kinematic viscosity variation, such as according to standard ISO 3104, of less than 14%, preferably of less than 10%.

13. A method according to claim 3, wherein in step a), a potassium permanganate to [compound (I)+compound (II)] molar ratio is used, ranging from 0.25:1 to 0.35:1, preferably from 0.28:1 to 0.33:1.
